(19) United States
(12) Patent Application Publication
(10) Pub. No.: US 2010/0221316 A1
(43) Pub. Date: Sep. 2, 2010

(54) Encapsulated Transfer Factor Compositions and Methods of Use

(76) Inventor: Joseph C. Ramaekers, Aptos, CA (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/631,745

(22) Filed: Dec. 4, 2009

Related U.S. Application Data
(63) Continuation of application No. 11/492,464, filed on Jul. 24, 2006, now abandoned.
Application No. 11/492,464 is a continuation-in-part of application No. 11/106,054, filed on Apr. 13, 2005, now abandoned.
Application No. 11/106,054 is a continuation of application No. 11/237,316, filed on Sep. 27, 2005, now abandoned.
Application No. 11/237,316 is a division of application No. 10/136,854, filed on Apr. 30, 2002, now Pat. No. 6,962,718.
Application No. 10/136,854 is a continuation-in-part of application No. 09/847,036, filed on Apr. 30, 2001, now Pat. No. 6,506,413.
(60) Provisional application No. 60/701,860, filed on Jul. 22, 2005.
(60) Provisional application No. 60/573,113, filed on May 20, 2004.
(60) Provisional application No. 60/649,363, filed on Feb. 1, 2005.

(51) Int. Cl. A61K 9/127; A61K 31/715

(52) U.S. Cl. 424/450; 514/54

(57) ABSTRACT

Methods for stress reduction comprising administering a transfer factor to an animal. The transfer factor can be combined with glucans, such as hybrid glucans and other components, to bring about stress reduction. The formulation is encapsulated when administered to adult ruminants.

This application claims benefit under 35 U.S.C. §119(e) of U.S. Provisional Application No. 60/701,860 filed Jul. 22, 2005, and is a continuation-in-part of application Ser. No. 11/106,054, filed Apr. 13, 2005, each of which is expressly incorporated by reference in its entirety.

### FIELD OF THE INVENTION

Methods for stress reduction comprising administering a transfer factor to an animal. The transfer factor can be combined with glucans, such as hybrid glucans and other components, to bring about stress reduction. The formulation is encapsulated when administered to adult ruminants.

### BACKGROUND OF THE INVENTION

Transfer factors which are produced by leucocytes and lymphocytes, are small water soluble polypeptides of between about 44 amino acids that stimulate or transfer cell mediated immunity from one individual to another and across species but do not create an allergic response. Since transfer factors are smaller than antibodies, they do not transfer antibody mediated responses nor do they induce antibody production. The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

Transfer factor has been described as an effective therapeutic for Herpes simplex virus (Viza, et al.), a treatment for acne blemishes, U.S. Pat. No. 4,435,384 and as a treatment against C. albicans (Khan et al.). Transfer factor has also been used to treat intestinal cryptosporidiosis in recipients treated with specific transfer factor (McMeeking, et al.). Still, et al. also showed that chicken pox infections were prevented by pretreatment of children treated with transfer factor from individuals that had chicken pox or who in other words had been sensitized to the varicella antigen. The antigen specific transfer factors are the most well studied and have been demonstrated to be able to convey the antigen recognition ability of the experienced donor to the naive recipient. It may be assumed that the individual or animal that is the source of the transfer factor has been sensitized to the antigen of interest. The term antigen is defined herein is anything that will initiate the cell mediated immune response. However, transfer factor as found in commercial bovine colostrum extract coming from a pool of animals (e.g., cows) contains the acquired immunity from all of the pool and therefore provides a type of generalized adoptive transfer of immunity. Transfer factors or transfer factor can be obtained from a dialyzable extract of the lyzed cells or from an extract of extracellular fluid containing transfer factor. Common sources of transfer factors are colostrums and ova. It is common practice to refer to preparations that contain transfer factor by the name of the active component (i.e., transfer factor or TF). Transfer factor extract containing transfer factors is also herein referred to as transfer factor. Transfer factor from bovine colostrum extract is defined as defatted water soluble material from colostrum that will pass through a nominal 10,000 molecular weight filter. The colostral derived transfer factor has been prepared with activity against various organisms including infectious bovine rhinotracheitis virus. One of the specific effects of transfer factor is a significantly increased natural killer (NK) cell activity. Natural killer cells provide protection against viruses as part of the innate immune defense system.

Although transfer factor is a polypeptide, it has been reported that it is surprising stable in the gastrointestinal tract. For example, Kirkpatrick compared oral versus parental administration of transfer factor in clinical studies. Kirkpatrick, Biotherapy, 9:13-16, 1996. He concluded that the results refute any arguments that the acidic or enzymatic environment of the gastrointestinal tract would prevent oral therapy using transfer factors.

When attempts were made to sequence TF, it was reported that an N-terminal end of the transfer factor peptide is resistant to sequential Edman degradation. Kirkpatrick, Molecular Medicine, 6(4):332-341 (2000).

Transfer factors have also been used successfully in compositions for treating animal diseases and syndromes including ruminants. See U.S. Patent Publication 2003/0077254, published Apr. 24, 2003.

Accordingly, transfer factor was believed to be stable in the gastrointestinal tract and rumen.

### SUMMARY OF THE INVENTION

The invention provides methods for reducing stress in ruminating and non-ruminating animals.

In one embodiment, the method comprises administering an encapsulated composition comprising transfer factor and glucan. Encapsulation protects transfer factor and glucan from inactivation in the gastrointestinal tract.

Such encapsulation is important especially in the case of ruminants where digestion within the rumen has been found to be problematic. Enhanced bioavailability has been demonstrated when a transfer factor is encapsulated and administered to ruminants. In preferred embodiments, the transfer factor and glucan are encapsulated by mixing with a hydrophobic substance or a lipid to form a coating around the transfer factor and glucan.

In the case of non-ruminating mammals, the transfer factor and glucan need not be encapsulated with a hydrophobic substance or lipid to form a coating around the formulation. The transfer factor and glucan can be administered separately or as a mixture. In some embodiments the transfer factor and/or glucan can be contained separately or together within a gel capsule. In other embodiments, the transfer factor and/or glucan can be contained within a medium which allows transport of the formulation past the stomach wherein the transfer factor and glucan are released in the small intestine.

In an alternative embodiment, transfer factor and glucan can be encapsulated with a hydrophobic substance or a lipid coating and used to reduce stress in non-ruminating mammals. A non-ruminating mammal is one that is monogastric.

In some instances, formulation without hydrophobic or lipid coating can be administered to newly born ruminates. The formulation bypasses the rumen via the esophageal groove.

Treatment with transfer factor and glucan results in (1) a decrease in cortisol levels as compared to control animals; (2) an increase in thyroid function as compared to control animals; and (3) an increase in insulin as compared to control animals. In addition the diurnal rhythm from cortisol and thyroid function is improved. Disruption of the cortisol diurnal rhythm is indicative of stress. A return to normal diurnal rhythm corollates with stress reduction in the treated animal.

In some embodiments, the glucan is a hybrid glucan. In some embodiments, the transfer factor is a targeted transfer factor.

In some embodiments, stress reduction occurs after the administration of transfer factor alone. Stress can arise from pathological and/or environmental conditions. In some instances, the method is useful for reducing environmental stress, i.e., stress that is not pathological, e.g., stress associated with Cushings disease.

The formulations for stress reduction may also include one or more of the following: Inositol hexaphosphate, Olive leaf extracts, Aloe extract powder, β-sitosterol, yeast extract, ascorbic acid, di-potassium phosphate, potassium chloride, magnesium sulfate, calcium pantothenate, vitamin E, vitamin C, vitamin A, vitamin D3, vitamin B1, vitamin B2, vitamin B12 and zinc, e.g., zinc proteinate.

### BRIEF DESCRIPTION OF THE FIGURES

FIG. 1 sets forth the results obtained using the encapsulated transfer factor formulation of Table 7. Morbidity was reduced from 15.5% to 3.1% while mortality was decreased from 5.5% to 0% when animals treated with encapsulated transfer factor are compared to controls that were not treated with transfer factor. In addition, the daily weight gain of the controls was 1.85 lbs/day versus 3.05 lbs/day for those animals treated with the encapsulated transfer factor formulation.

FIG. 2 is a second study involving the use of the encapsulated transfer factor formulation of Table 7 in a different field study using high stress cattle. In this study, the morbidity of the animals was reduced from 83% to 2.6% and the mortality reduced from 24% to 0% in those animals treated with encapsulated transfer factor formulation as compared to control that did not receive transfer factor. In addition, the control population had a weight increase of 0.9 lbs/day as compared to 3.1 lbs/day for those animals treated with the encapsulated transfer factor formulation.

FIG. 3 sets forth the blood cortisol levels in study calves and control calves as a function of time.

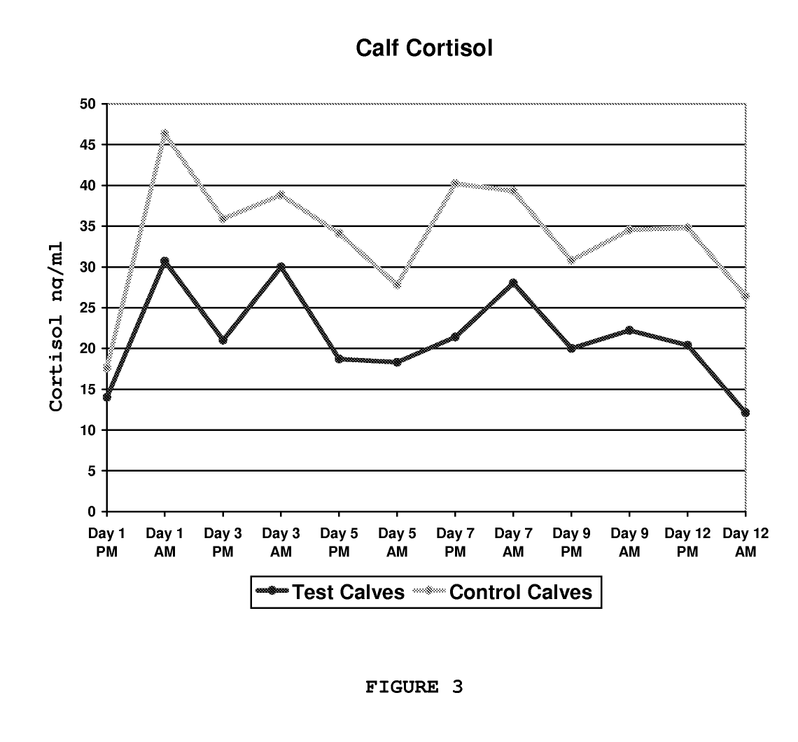

FIG. 4 sets forth the blood insulin levels in study calves and control calves as a function of time.

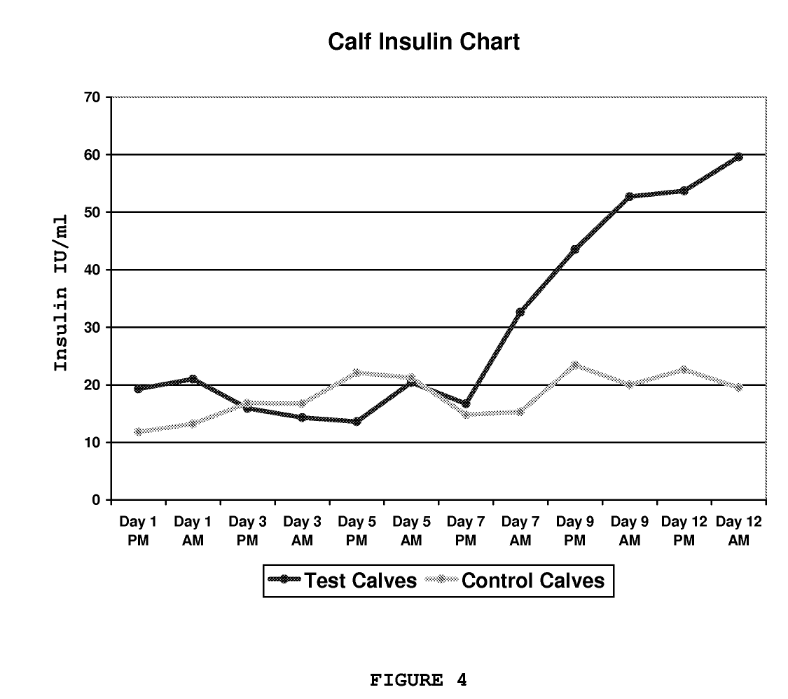

FIG. 5 is a bar graph showing the average cortisol and insulin levels for study calves and control calves.

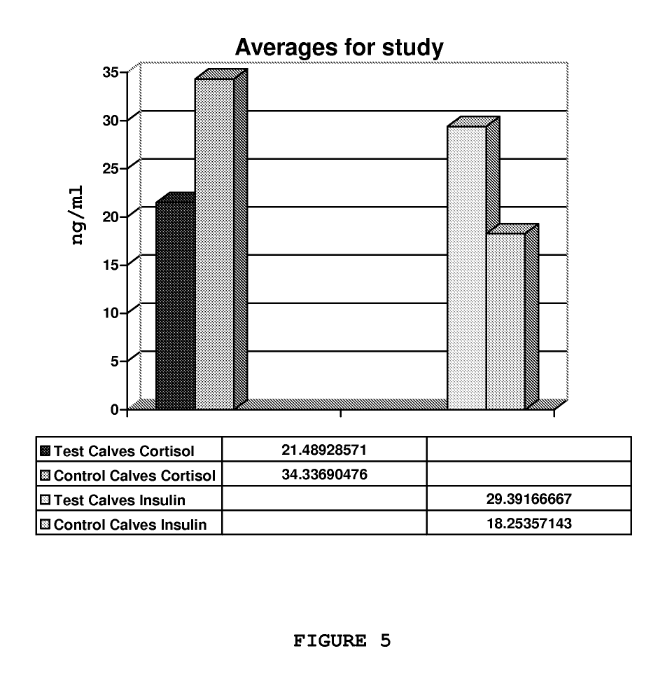

FIG. 6 is a bar graph showing the average weight gain for study calves and control calves over a 60-day period.

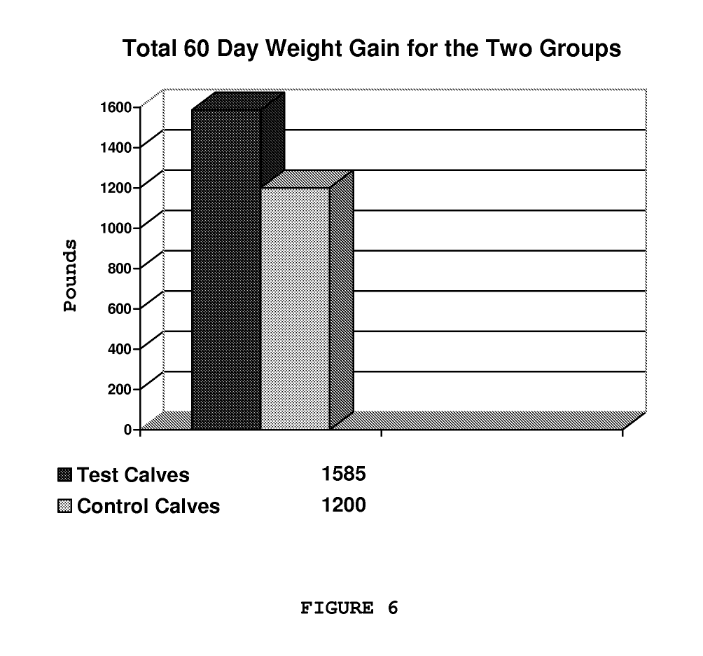

### DETAILED DESCRIPTION OF THE INVENTION

Encapsulated formulations of the invention contain encapsulated transfer factor and/or encapsulated glucan, including hybrid glucans. The transfer factor and/or glucan can be individually encapsulated or encapsulated as a mixture. Alternatively, the entire formulation can be encapsulated.

Various forms of transfer factor may be used in accordance with this invention. They include excreted transfer factor released from transfer factor containing cells such as lymphocytes, leukocytes and ova, and collected from extracellular fluids such as colostrums and blood. Another form includes preexcreted transfer factor found within the cell or on the cell surface. Substantially purified transfer factor originating from leukocytes, clostrum or ova and having a molecular weight of less than 10,000 daltons and a specific activity of at least 5000 units per adsorbance unit at 214 nanometers, may also be used. The transfer factor used in the Examples of this invention and referred to in the following Tables and further referred to in the rest of the detailed description is extracted from colostrum collected from a general pool of lactating cows and eggs. The transfer factor, as used in the Examples, Tables and the following description, is further defined as defatted water soluble material from bovine colostrum that will pass through a nominal 10,000 molecular weight filter. Though bovine colostral derived transfer factor was used to develop the formulations of this invention, it is well known to anyone skilled in the art that other kinds and sources of transfer factor could be used.

Alternative sources of transfer factor include, but are not limited to, avian transfer factor, ova transfer factor, and transfer factor isolated from colostrum collected from non-bovine animals such as goats, pigs, horses and humans. In addition, combinations of transfer factors from any number of sources may be used in the formulations of the instant invention. Transfer factor may also be derived from recombinant cells that are genetically engineered to express one or more transfer factors or by clonal expansion of leukocytes.

Alternative kinds of transfer factor include, but are not limited to, targeted transfer factors. Target transfer factors include transfer factor collected from sources which have been exposed to (1) one or more viral or otherwise infectious organisms; (2) one or more antigens that produce an immune response; or (3) a combination of organisms and antigens. Examples of such viral or other infectious organisms include Herpes Simplex Virus 1, Herpes Simplex Virus 2, H. Pylori, Champhobactor and Chlamydia, Bovine Rhinotracheitis Virus, Parainfluenza, Respiratory Syncytial Virus Vaccine, modified live virus, Campylobacter Fetus, Leptospira Canicola, Grippotyphosa, Hardjo, Leterohaemorrhagiae, Pomona Bacterin, Bovine Rota-Coronavirus, Escherichia Coli Bacterin, Clostridium Chauvoei, Septicum, Haemolyticum, Novy, Sordellii, Perfringens Types C & D, Bacterin, Toxoid, Haemophilus Somnus, Pasteurella Haemolytica, Multocida Bacterin. However, one of skill in the art would readily recognize that a wide variety of other viral and otherwise infectious organisms can find use in the instant invention. Examples include those set forth in Appendix I and Appendix II.

Table 1 sets forth typical components of Montmorillonite.

Tables 2-6 set forth transfer factor formulations that have been used to treat various animals and pathologies. In each case, the transfer factor is not encapsulated as set forth herein. However, the transfer factor in each of these formulations can be readily encapsulated with a hydrophobic or lipid coating prior to admixture with the other components of the formulation.

Table 2, shows a breakdown of a formulation of transfer factor nutraceuticals and carriers for treating Cushing syndrome, Cushings disease, adenomas, onchocerciasis, hypothyroidism or EPM. In Table 2 and all the other tables references to “lb” (pounds) means pounds of body weight.

Columns 2, 3 and 4 of Tables 2-6 show the approximate high, low and preferred amounts, respectively, of the formulation components, in amounts per body weight, to be given to an animal in a single dosage. The formulations in Tables 3 and 4 are very similar to the formulation of Table 2 but they are specialized for dogs and cats respectively. The formulation represented in Table 2 is designed primarily for livestock. The 5 ounces of the formula listed in column 5 is designed to be given to a 1000 pound animal but that will vary and could be given to a 500 pound animal in some cases. The average horse is around 1000 pounds. The 28.3 gm dosage in Table. 3 is calculated for a dog weighing about 100-200 pounds but that dosage may also be given to a 15 pound dog. The 2.2 gm formula in Table 4 is for a cat weighing around 15 pounds. However, since these formulas are comprised of nutraceuticals and transfer factor, one skilled in the art will recognize that the ranges are not certain and as critical as the ranges for allopathic drugs.

Further, the formulations in Tables 2-4 are designed to treat mainly chronic diseases, the formulation in Table 5 is designed for mainly acute diseases and the formulation in Table 6 is for both acute and chronic diseases. All the formulations may be given in megadoses to achieve an acute response.

Table 7 provides an encapsulated transfer factor formulation for treating pathologies. This transfer factor formulation includes at least encapsulated transfer factor derived from both bovine and avian sources, and/or one or more of hybrid glucans. It is preferred that the glucan portion of this formulation also be encapsulated. Other components include zinc proteinate, targeted avian transfer factors, β-sitosterol, inositol hexaphosphate (IP6), olive leaf extract, aloe extract powder, probiotics, B. subtlis, B. longum, B. thermophilium, L. acidophilus, E. faecium, and S. cerevisia. In a preferred embodiment, all of the foregoing are included in this transfer factor formulation.

In preferred encapsulation embodiment, transfer factor is present in the formulation in the amount of 10 mg to 12 gm/oz, more preferably 100 mg to 6 gm/oz and most preferably 10 mg to 3 gm/oz.

The transfer factor is encapsulated with a hydrophobic or lipid coating that is preferably between 25% and 150 wt/% of the transfer factor, about 50-150 wt/% and about 75-125 wt/% with an equal weight being most preferred.

In a preferred embodiment the hybrid glucans used in the invention are present in, or derived from, hybrid strains of Cordyceps and in particular Cordyceps sinensis. One technique to induce the hybridization of Cordyceps involves plating two different strains or species on a single agar plate which has been inoculated with rattlesnake venom as described in detail in Examples 17 and 18. As described, the snake venom functions to weaken the cell walls of the Cordyceps strains/species which allows for the exchange of nuclear material between the strains/species as they grow nearer to each other. In a preferred embodiment, the hybrid strain producing the hybrid glucans of the invention is Cordyceps sinensis Alohaensis which is available from Pacific Myco Products, Santa Cruz, Calif.

There are a number of different Cordyceps sinensis strains and due to their variable asexual mycelial growth forms they have been considered to be different species by many taxonomists. A non-exhaustive list of strains includes: Paecilomyces hepiali Chen, Cephalsporim sinensis, Paecilomyces sinensis Cn80-2, Scydalilum sp., Hirstutella sinenis, Mortierella hepiali, Chen Lu, Topycladium sinensis, Scytalidium hepiali, G. L. Li. Preferred embodiments of the instant invention make use of hybrid glucans from hybrids of one or more of these different strains, however, the invention may alternatively preferentially include glucans from non-hybridized strains. Alternative embodiments utilize the whole hybrid Cordyceps, e.g., Cordyceps sinensis Alohaensis. Hybrid glucans also include those obtained by crossing sources of feed, e.g., oats, etc.

When glucans or hybrid glucans are used, the formulation preferably contains 10 mg to 18 gm of whole organism/oz, more preferably 100 mg to 10 gm of whole organism/oz and most preferably 100 mg to 5 gm of whole organism/oz.

Equivalent amounts of purified or partially purified glucan or hybrid glucans as well as the nucleosides associated therewith (e.g., Cordycepin (3′deoxyadenosine), adenosine and N⁶-(2 hydroxyethyl)-adenosine) can also be used.

As with encapsulated transfer factor, it is preferred that the amount of hydrophobic or lipid coating be between about 25% and 150 wt/% of the hybrid glucan, about 50-150 wt%, or about 75-125 wt/% with an equal weight being most preferred.

Other components of the formulation may also be encapsulated. For example, IP6β-sitosterol, olive leaf extract, aloe extract matter and/or vitamin C can be individually encapsulated or may be combined with one or more components prior to encapsulation. In preferred embodiments, IP6 is present at between 10 mg and 3 gm/oz, or one preferably between 100 mg and 2 gm/oz, and most preferably between 100 mg and 1 gm/oz. The β-sitosterol is preferable in the amount of between 10 mg and 3 gm/oz, or preferably between 100 mg and 2 gm/oz, and most preferably between 100 mg and 1 gm/oz. Olive leaf extract is preferably present in the amount of 2 mg to 2 gm/oz, more preferably between 5 mg and 1 gm/oz, and most preferably between 5 mg and 500 gm/oz. Aloe extract is preferably present at between 2 mg and 1000 mg, more preferably between 5 and 500 mg/oz, and most preferably between 5 and 250 mg/oz. Vitamin C may be present at between 10 mg/oz and 10 gm/oz, or preferably between 100 mg and 8 gm/oz, and most preferably between 100 mg and 5 gm/oz.

The amount of transfer factor and/or glucan used in the formulation or the amount of formulation administered will vary depending upon the severity of the clinical manifestations presented. In addition, the amount of transfer factor administered to a recipient will vary depending upon the species from the transfer factor is derived as compared to the species of the recipient. It has been observed that transfer factor derived from bovine species administered to cattle is more efficacious than transfer factor from another species such as avian species. Accordingly, when the source of the transfer factor and recipient are different species, it is preferred that the amount of transfer factor be increased.

Administration of a formulation of an encapsulated transfer factor with zinc and at least one essential fatty acid is expected to result in at least a partially effective treatment of Cushings syndrome, Cushings disease, adenomas and other benign tumors, onchocerciasis, hypothyroidism or EPM. The treatment is more effective as other nutraceuticals listed in Table 2 are added. The dosage is in milligrams per pound unless otherwise stated. The amounts of the components present in a 5 ounce transfer factor formulation containing the other preferred nutraceuticals is shown in column 5 of Table 2.

Encapsulated transfer factor at a dosage of about 0.75 mg/lb transfer factor in combination with about 0.49 mg/lb zinc and 20.57 mg/lb of canola oil, safflower oil or flax oil, sources of essential fatty acids (i.e., 3, 6, 9 omega fatty acids), given once daily to an animal suffering from Cushings syndrome, Cushings disease, adenomas or other benign tumors, onchocerciasis, hypothyroidism or equine protozoal myelytis should result in approximately a 30% to 50% reduction in the size of the benign tumors and/or the symptoms of these listed diseases. All of these components should of course be pharmaceutically acceptable to the animal receiving them.

A combination of Vitamin C at about 2.16 mg/lb and 2.29 mg/lb of yeast in combination with the above listed transfer factor and other fatty acid nutraceuticals should results in approximately a 40% to 50% reduction in the size of benign tumors and /or symptoms of the above listed diseases.

It is preferred in all formulations of the invention that the metal nutraceuticals are proteinated because these forms are easier for the animal to digest and also because the proteinate forms are more stable to pH. The nutraceutical components in the formulations in Tables 2-7 are the active components for treating the various described diseases and syndromes. The fillers and carriers are included to make the formulations more palatable to the animal and also to help preserve the mixture. These include silicon dioxide, maltodextrin, soy and peanut flour, peanut oil, dextrose, whey, spices and flavorings. Mixed tocopherols and choline chloride are nutraceuticals but the effective results described herein can still be achieved by deleting these two components from the formulations.

Previous use of non-encapsulated transfer factor in ruminants, e.g., cows, produced significant beneficial results. See, e.g. U.S. Patent Publication 2003/0077254, published Apr. 24, 2003 incorporated herein by reference in its entirety. Subsequently, it was discovered that transfer factor was not stable by oral administration in a stressed population of cattle. After discovering that transfer factor is inactivated in vitro in the presence of rumen fluid and flora, it was determined that prior success with transfer factor in ruminants was due to the presence of the esophageal groove. When not stressed, the esophageal groove provides partial bypass of the rumen. However, in a stressed population the esophageal groove closes and shunts the transfer factor formulation into the rumen. It was discovered that encapsulating transfer factor and/or glucans with a hydrophobic substance or a lipid to form an encapsulated formulation is sufficient to provide substantial by-pass of (e.g., 85%) of the rumen even in a stressed population.

A variety of other methods for rumen by-pass are known. In one embodiment, the encapsulated or non-encapsulated formulation is directly injected (subcutaneously, intramuscularly, or intravenously) to by-pass not only the rumen but also the entire digestive system. Similarly, intravaginal, intrarectal or other direct administration to mucus membranes, such as the eye subconjunctival, by-pass the digestive system and the rumen in particular. Alternatively, the formulation can be mixed with various solvents which allow for direct skin absorption. Furthermore, methods are known in the art to stimulate opening of the esophageal groove in various ruminants and such opening allows for immediate passage of an orally administered formulation to the gastrointestinal tract, by-passing the rumen.

In a particularly preferred embodiment, rumen by-pass is facilitated by use of an encapsulated transfer factor formulation.

The encapsulated transfer factor and/or encapsulated glucan formulation can be produced in a variety of ways. In a preferred embodiment, each of the transfer factor and/or glucan in the formulation is encapsulated as described in U.S. Pat. Nos. 5,190,775, 6,013,286 and U.S. Application 2003/0129295, each of which is incorporated herein by reference in their entirety. In brief, the methods described in the cited patents and application center on the use of a hydrophobic or lipid coating that provides protection from the degredative nature of the rumen, in combination with an additional surfactant coating to inhibit floating of the encapsulated formulation in order to facilitate passage of the formulation out of the rumen and further through the digestive system. Preferred examples of hydrophobic coatings include, but are not limited to, plant oils and hydrogenated plant oils, each derived or made from palm, palm kernel, cottonseed, soybean, corn, peanut, babassu, sunflower or safflower oil and mixtures thereof. In addition, such coatings may be mixed with wax, such as, but not limited to, beeswax, petroleum wad, rice bran wax, castor wax, microcrystalline wax, and mixtures thereof. Preferred examples of surfactants include, but are not limited to, polysorbate 60, polysorbate 80, propylene glycol, sodium dioctylsulfosuccinate, sodum lauryl sulfate, lactylic esters of fatty acids, polyglycerol esters of fatty acids, and mixtures thereof.

Such encapsulated formulations have a variety of benefits in addition to their role in rumen by-pass. First, encapsulation protects the formulation from degradation and provides for a significantly longer shelf-life. Such encapsulated formulations can withstand heating to temperatures of more than 135° F. that are necessary for a number of production processes including pelleting for animal feed or processing for human consumption. Encapsulation also removes bitterness and odors normally present in formulations, and thus greatly increases palatability. Encapsulation also allows flexibility in the formulation so that the fragile components do not interact with harsh minerals, salts or variable pH.

Due to the increases in shelf-life, thermal stability, palatability and flexibility, encapsulated formulations such as encapsulated transfer factor formulation are preferred for human and animal consumption. Preferred embodiments for human consumption include, but are not limited to incorporation of encapsulated transfer factor formulations in processed foods such as cereals, snacks, chips, or bars. Preferred embodiments for animal consumption include, but are not limited to, encapsulated transfer factor formulations admixed in feed pellets, salt licks, molasses licks or other processed feed products.

The encapsulated transfer factor formulations find use in increasing food conversion efficiency. Food conversion efficiency is the rate at which an organism can convert food to body mass, and is also known in the cattle industry as feed conversion efficiency. Encapsulated transfer factor formulations have been successfully used to increase the body weight of cattle at an enhanced rate as compared to non-treated cattle, even in situations where the treated cattle are diseased. Accordingly, the encapsulated formulations are not limited to prophylaxis and treatment of pathologies, but find use in other aspects of overall organismal health and development.

The encapsulated transfer factor formulations of the present invention include pharmaceutical compositions suitable for administration. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the pharmaceutical compositions are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

A daily dosage of 141 mg per pound of body weight of any of the formulations in column 5 of Tables 2, 3 or 4, for 14 days has been successful in treating feline pneumonitis, feline leukemia, feline autoimmune dysfunction, feline flea bit dermatitis, feline hyperthyroidism, feline viral infection, feline ulcerations, feline bacterial infection, canine flea bite dermatitis, canine Cushings disease, malignant tumors, canine autoimmune dysfunctiiion, canine viral and bacterial infection. These treatments for the most part have resulted in complete cures. The use of encapsulated transfer factor in these formulations is expected to produce the same or better results.

Administering a formulation comprising all of the nutraceuticals in Table 2 at the preferred dosage to an animal with benign tumors resulted in about a 60% reduction in the size of the benign tumors and about a 90% reduction in the symptoms exhibited by the animal suffering the above listed diseases and syndromes. The use of encapsulated transfer factor in these formulation is expected to produce the same or better results.

Administration of all of the nutraceuticals in Table 2 at the low dosage in column 3 of those tables results in about a 7% to 100% reduction in the size of the tumors and/or a 30% to 100% reduction in the symptoms exhibited by the animal suffering from those diseases or syndromes. The use of encapsulated transfer factor in these formulations is expected to produce the same or better results.

The stress formulation in Table 5 is also used to treat numerous animal diseases and syndromes and as stated previously, mainly their acute stages. This formulation is also water soluble so it can be given in the animals drinking water. A mixture of about 0.75 mg/lb transfer factor and about 1.42 mg/lb lactobacillus acidophilus 10⁹ colony forming units (CFU) given twice daily will result in at least a 30% reduction in clinical symptoms resulting from strangles, dust cough, hypothyroidism and lymphopenia. The same dosage given to young calves will also reduce morbidity by about 30%. The addition of ionic salts or chelates of calcium, magnesium sodium and potassium twice daily in amounts approximating those in column 4 of Table 5 to the above amounts of transfer factor and lactic acid generating bacterial results in a 40% reduction in clinical symptoms of the above mentioned diseases. The addition of about 0.482 mg/lb of citric acid to the above formulation results in about a 45% reduction in the symptoms of the above mentioned diseases. Further addition of Vitamins A, B₂, B₆, B₁₂, C and E, and thiamine results in a 50% reduction in the symptoms of these diseases. The stress formulations given once or twice a day in the dosage presented in column 4 of Table 5 will cure or at least treat and reduce the symptoms of autoimmune dust cough, diarrhea from viral etiology, abscessation, in strangles, snotty nose in strangles, acute viremia in swine, scratches in the horse, hypersensitivity from scratches and onchoceriasis, PURRS, BRD, calf dysentery, coliform infections, Rhodococcus infections, Clostidium infections, circo virus in birds, and pnemonitis in cats. A combination of transfer factor and lactic acid producing bacteria or this combination further combined with yeast as shown in Table 5 will also treat these diseases but to a lesser extent. The use of encapsulated transfer factor is expected to produce the same or better results.

The stress formulation as shown in Table 5 given once or twice daily will also increase the weight gain and feed efficiency of livestock. The weight gain will increase by at least 8%. A combination of transfer factor and lactic acid producing bacteria or this combination further combined with yeast as shown in Table 5 will also increase weight gain but to a lesser extent. The use of encapsulated transfer factor is expected to produce the same or better results. In a preferred embodiment, 2 gm of encapsulated hybrid glucan containing 1 gm of hybrid glucan is used.

Table 6 shows a breakdown of a performance formulation of transfer factor and nutraceuticals for treating and curing numerous diseases such as arthritis, laminitis, inflammation and malignant tumors. These diseases may also be treated with a combination of transfer factor and super oxide dismutase; transfer factor and glucosamine salts; transfer factor, glucosamine salts and super oxide dismutase; transfer factor, glucosamine salts, super oxide dismutase and glycine; transfer factor, glucosamine salts, super oxide dismutase, glycine and methyl sulfonyl methane; transfer factor, glucosamine salts, super oxide dismutase, glycine, methyl sulfonyl methane and octocosonol or transfer factor, glucosamine salts, super oxide dismutase, glycine, methyl sulfonyl methane, octocosonol and montmorillinite.

Table 7 shows a formula containing transfer factor and glucan both hybridized and non-hybridized.

Any of the aforementioned formulations can be incorporated into an encapsulated formula.

TABLE 1
Montmorillonite Components
Average Nutrient Content Per Ounce
(1 Tablespoon = ~0.36 oz.)
(mg)

| | | | |
|---|---|---|---|
| Silicon | 6933 | Tungsten | 0.218 |
| Aluminum Silica | 2505 | Vanadium | 0.215 |
| Sodium Chloride | 1320 | Ruthenium | 0.210 |
| Potassium | 1293 | Baron | 0.189 |
| Protein | 1116 | Bromine | 0.140 |
| Calcium | 1104 | Cobalt | 0.129 |
| Sulfur | 431 | Selenium | 0.110 |
| Iron | 431 | Syprosium | 0.107 |
| Magnesium | 224 | Fluorine | 0.102 |
| Chlorine | 164 | Scandium | 0.0997 |
| Titanium | 61.9 | Samarium | 0.0943 |
| Carbon | 48.2 | Nobelium | 0.0754 |
| Sodium | 37.2 | Copper | 0.0593 |
| Barium | 10.5 | Praseodymium | 0.0539 |
| Phosphate | 8.62 | Erbium | 0.0539 |
| Strontium | 6.46 | Hafnium | 0.0539 |
| Cesium | 4.93 | Ytterbium | 0.0377 |
| Manganese | 4.04 | Lithium | 0.0377 |
| Thorium | 2.69 | Yttrium | 0.0323 |
| Uranium | 2.69 | Holmium | 0.0296 |
| Arsenic | 1.97 | Cadmium | 0.0296 |
| Chromium | 1.89 | Palladium | 0.0189 |
| Molybdenum | 1.64 | Terbium | 0.0161 |
| Nickel | 1.62 | Thulium | 0.0161 |
| Iodine | 1.28 | Gold | 0.0161 |
| Lead | 1.17 | Tantalum | 0.0135 |
| Cerium | 1.08 | Iridium | 0.0135 |
| Rubidium | 0.983 | Lutetium | 0.0108 |
| Antimony | 0.781 | Europium | 0.0108 |
| Gallium | 0.673 | Rhodium | 0.0108 |
| Germanium | 0.673 | Tin | 0.0108 |
| Neodymium | 0.539 | Silver | 0.00808 |
| Zinc | 0.539 | Indium | 0.00808 |
| Lanthanum | 0.486 | Oxygen | 0.00539 |
| Bismuth | 0.385 | Mercury | 0.00269 |
| Zirconium | 0.269 | Tellurium | 0.00269 |
| Rhenium | 0.269 | Beryllium | 0.00269 |
| Thallium | 0.269 | | |

TABLE 2
Premix Formulation
(Amounts in mg/lb of body weight unless otherwise stated)

| | | | | | Dosage: mg/5 oz. |
|---|---|---|---|---|---|
| Component | High | | Low | Preferred | of formula |
| l-Arginine | 0.5 | | 0.005 | 0.05 | 50.00 |
| *Lacto yeast (4.9% of blend) | 69.51 | | 0.6951 | 6.91 | 6951.88 |
| Montmorillinite | 1 | gm/lb | 0.24118 | 2.4118 | 2411.88 |
| Canola oil (14.5% mix) | 1.5 | gm/lb | 2.05 | 20.571 | 20571.88 |
| Safflower oil (14.5% mix) | 1.5 | gm/lb | 2.05 | 20.57 | 20571.88 |
| Flax seed oil (55% Alpha Linolenic | 1.5 | gm/lb | 2.05 | 20.571 | 1418.75 |
| Acid) (1.0% mix) | | | | | |
| Phosphorous (Monosodium | 15.750 | gm | 0.0525 | 5.08 | 5080.00 |
| phosphate) 12% | | | | | |
| Calcium carbonate 8.5% | 13.68 | gm | 0.0485 | 4.88 | 4880.00 |
| (38% calcium) | | | | | |

| | | | | |
|---|---|---|---|---|
| Methyl sulfonyl methane | 20 | 0.02 | 2 | 2000.00 |
| Transfer factor | 50.00 | 0.05 | 0.75 | 750.00 |
| Vitamin C (ascorbic acid) | 21.62 | 0.2162 | 2.162 | 2162.50 |
| d-Biotin (Vitamin H 2%) | 9.73 | 0.000973 | 0.00973 | 10.00 |
| Vitamin D₃ | 29.16 IU | 0.7298 IU | 7.298 IU | 7298.38 IU |
| Vitamin B₁₂ | 0.092 | 0.000092 | 0.00092 | 0.92 |
| Folic Acid | 1 | 0.001006 | 0.01006 | 10.06 |
| Niacinimide | 12 | 0.012157 | 0.12157 | 121.57 |
| Pantothenic acid (d-Calcium | 0.324 | 0.0108 | 0.108 | 108.00 |
| Pantothenate) 91.6% | | | | |
| Vitamin B₆ (Pyridine Hcl) 82.3%) | 1.158 | 0.001158 | 0.01158 | 11.58 |
| Vitamin A (Retinol Palmitate) 650M | 600 IU | 4.02 IU | 40.212 IU | 40232.50 IU |
| IU/g feed grade | | | | |
| Vitamin B₂ | 0.0554 | 0.002776 | 0.02776 | 27.76 |
| Thiamine (Mononitrate) 83% | 3.09 | 0.00308 | 0.0308 | 30.80 |
| Vitamin E | 72.9 IU | 0.0729 IU | 0.729 IU | 729.42 IU |
| Vitamin K | 1 | 0.0007 | 0.007 | 7.00 |
| Cobalt (Proteinate) 5% | 0.00043 | 0.000043 | 0.00043 | 0.43 |
| Copper (Proteinate) 10% | 0.56 | 0.0112 | 0.112 | 112.00 |
| Iodine (Potassiumiodide) 98% | 0.005 | 0.000053 | 0.00053 | 0.53 |
| Iron (Proteinate) 15% | 3.31 | 0.0331 | 0.331 | 331.16 |
| Magnesium (Oxide) 58% | 10 | 0.04 | 0.4 | 400.00 |
| Manganese (Proeinate) 15% | 1.65 | 0.04 | 0.4 | 332.10 |
| Molybdenum (Sodium Molybdate | 0.05 | 0.001 | 0.01 | 10.00 |
| Dihydrate) 39% | | | | |
| Selenium (Sodium Selenite) 44.8% | 0.00162 | 0.000081 | 0.00081 | 1.00 |
| Zinc (Proteinate) 15% | 50 | 0.04987 | 0.4987 | 498.72 |
| l-Lysine (Mono HCl) | 8.41 | 0.0841 | 0.841 | 841.57 |
| d,l-Methionine | 11.03 | 0.1103 | 1.103 | 1103.86 |
| Mixed Tocopherols | | | | 300.00 |
| Choline Chloride | | | | 2434.00 |
| Sipernat 50 (Silicon dioxide) | | | | 12768.75 |
| Lodex-5 (maltodextrin) | | | | 7519.38 |
| Soy flour (17.5% mix) | | | | 24828.13 |
| Sweet whey | | | | 996.00 |
| BF70 spice | | | | 146.00 |
| Dextrose powder | | | | 750.00 |

*Lactic acid generating bacteria is two-thirds of component and yeast is one-third; lactic acid generating bacteria is 500,000,000 CFU/gm, yeast (e.g., “Saccharamyces”) 250,000,000 CFU/gm

TABLE 3
Canine Premix Formulation
(Amounts in mg/lb of body weight unless otherwise stated)

| | | | | | Dosage: mg/oz |
|---|---|---|---|---|---|
| Component | High | | Low | Preferred | of formula |
| l-Arginine | 0.5 | | 0.005 | 0.05 | 10.00 |
| *Lacto yeast (4.9% of blend) | 69.51 | | 0.6951 | 6.91 | 1390.38 |
| Montmorillinite | 1 | gm/lb | 0.24118 | 2.4118 | 482.20 |
| Canola oil (14.5% mix) | 1.5 | gm/lb | 2.05 | 20.571 | 3887.00 |
| Safflower oil (14.5% mix) | 1.5 | gm/lb | 2.05 | 20.57 | 3887.00 |
| Flax seed oil (55% Alpha | 1.5 | gm/lb | 2.05 | 20.571 | 240.00 |
| Linolenic Acid) (1.0% mix) | | | | | |
| Phosphorous (Monosodium | 15.750 | gm | 0.0525 | 5.08 | 1010.00 |
| phosphate) 12% | | | | | |
| Calcium carbonate 8.5% | 13.68 | gm | 0.0485 | 4.88 | 977.00 |
| (38% calcium) | | | | | |

| | | | | |
|---|---|---|---|---|
| Methyl sulfonyl methane | 20 | 0.02 | 2 | 400.00 |
| Transfer factor | 50.00 | 0.05 | 2.50 | 500.00 |
| Vitamin C (ascorbic acid) | 21.62 | 0.2162 | 2.162 | 432.50 |
| d-Biotin (Vitamin H 2%) | 9.73 | 0.000973 | 0.00973 | 2.00 |
| Vitamin D₃ | 29.16 IU | 0.7298 IU | 7.298 IU | 1459.68 IU |
| Vitamin B₁₂ | 0.092 | 0.000092 | 0.00092 | 0.18 |
| Folic Acid | 1 | 0.001006 | 0.01006 | 2.16 |
| Niacinimide | 12 | 0.012157 | 0.12157 | 24.31 |
| Pantothenic acid (d-Calcium | 0.324 | 0.0108 | 0.108 | 21.60 |
| Pantothenate) 91.6% | | | | |
| Vitamin B₆ (Pyridine Hcl) 82.3%) | 1.158 | 0.001158 | 0.01158 | 2.32 |
| Vitamin A (Retinol Palmitate) | 600 IU | 4.02 IU | 40.212 IU | 8046.50 IU |
| 650M IU/g feed grade | | | | |
| Vitamin B₂ | 0.0554 | 0.002776 | 0.02776 | 5.55 |
| Thiamine (Mononitrate) 83% | 3.09 | 0.00308 | 0.0308 | 0.16 |
| Vitamin E | 72.9 IU | 0.0729 IU | 0.729 IU | 145.88 IU |
| Vitamin K | 1 | 0.0007 | 0.007 | 1.40 |
| Cobalt (Proteinate) 5% | 0.00043 | 0.000043 | 0.00043 | 0.086 |
| Copper (Proteinate) 10% | 0.56 | 0.0112 | 0.112 | 22.40 |
| Iodine (Potassiumiodide) 98% | 0.005 | 0.000053 | 0.00053 | 0.106 |
| Iron (Proteinate) 15% | 3.31 | 0.0331 | 0.331 | 66.23 |
| Magnesium (Oxide) 58% | 10 | 0.04 | 0.4 | 80.00 |
| Manganese (Proeinate) 15% | 1.65 | 0.04 | 0.4 | 66.42 |
| Molybdenum (Sodium Molybdate | 0.05 | 0.001 | 0.01 | 2.00 |
| Dihydrate) 39% | | | | |
| Selenium (Sodium Selenite) | 0.00162 | 0.000081 | 0.00081 | 0.20 |
| 44.8% | | | | |
| Zinc (Proteinate) 15% | 50 | 0.04987 | 0.4987 | 99.74 |
| l-Lysine (Mono HCl) | 8.41 | 0.0841 | 0.841 | 176.91 |
| d,l-Methionine | 11.03 | 0.1103 | 1.103 | 220.07 |
| Mixed Tocopherols | | | | 60.00 |
| Choline Chloride | | | | 486.80 |
| Sipernat 50 (Silicon dioxide) | | | | 2553.35 |
| Lodex-5 (maltodextrin) | | | | 1508.87 |
| Peanut oil | | | | 496.56 |
| Soy flour (17.5% mix) | | | | 4965.02 |
| Peanut flour | | | | 4965.02 |
| Sweet whey | | | | 400.00 |
| BF70 spice | | | | 29.20 |
| Dextrose powder | | | | 500.00 |

*Lactic acid generating bacteria is two-thirds of component and yeast is one-third; lactic acid generating bacteria is 500,000,000 CFU/gm, yeast (e.g., “Saccharamyces”) 250,000,000 CFU/gm

TABLE 4
Feline Premix Formulation
(Amounts in mg/lb of body weight unless otherwise stated)

| | | | | | Dosage: mg/2.2 gm |
|---|---|---|---|---|---|
| Component | High | | Low | Preferred | of formula |
| l-Arginine | 0.5 | | 0.005 | 0.05 | 0.78 |
| *Lacto yeast (4.9% of blend) | 69.51 | | 0.6951 | 6.91 | 108.42 |
| Montmorillinite | 1 | gm/lb | 0.24118 | 2.4118 | 37.00 |
| Canola oil (14.5% mix) | 1.5 | gm/lb | 2.05 | 20.571 | 323.25 |
| Safflower oil (14.5% mix) | 1.5 | gm/lb | 2.05 | 20.57 | 323.25 |
| Flax seed oil (55% Alpha | 1.5 | gm/lb | 2.05 | 20.571 | 22.13 |
| Linolenic Acid) (1.0% mix) | | | | | |
| Phosphorous (Monosodium | 15.750 | gm | 0.0525 | 5.08 | 78.70 |
| phosphate) 12% | | | | | |
| Calcium carbonate 8.5% | 13.68 | gm | 0.0485 | 4.88 | 75.69 |
| (38% calcium) | | | | | |

| | | | | |
|---|---|---|---|---|
| Methyl sulfonyl methane | 20 | 0.02 | 2 | 31.20 |
| Transfer factor | 50.00 | 0.05 | 16.00 | 250.00 |
| Vitamin C (ascorbic acid) | 21.62 | 0.2162 | 2.162 | 33.73 |
| d-Biotin (Vitamin H 2%) | 9.73 | 0.000973 | 0.00973 | 0.156 |
| Vitamin D₃ | 29.16 IU | 0.7298 IU | 7.298 IU | 113.90 IU |
| Vitamin B₁₂ | 0.092 | 0.000092 | 0.00092 | 0.014 |
| Folic Acid | 1 | 0.001006 | 0.01006 | 0.168 |
| Niacinimide | 12 | 0.012157 | 0.12157 | 1.90 |
| Pantothenic acid (d-Calcium | 0.324 | 0.0108 | 0.108 | 1.68 |
| Pantothenate) 91.6% | | | | |
| Vitamin B₆ (Pyridine Hcl) 82.3%) | 1.158 | 0.001158 | 0.01158 | 0.18 |
| Vitamin A (Retinol Palmitate) | 600 IU | 4.02 IU | 40.212 IU | 627.60 IU |
| 650M IU/g feed grade | | | | |
| Vitamin B₂ | 0.0554 | 0.002776 | 0.02776 | 0.43 |
| Thiamine (Mononitrate) 83% | 3.09 | 0.00308 | 0.0308 | 0.48 |
| Vitamin E | 72.9 IU | 0.0729 IU | 0.729 IU | 11.38 IU |
| Vitamin K | 1 | 0.0007 | 0.007 | 0.11 |
| Cobalt (Proteinate) 5% | 0.00043 | 0.000043 | 0.00043 | 0.006 |
| Copper (Proteinate) 10% | 0.56 | 0.0112 | 0.112 | 1.75 |
| Iodine (Potassiumiodide) 98% | 0.005 | 0.000053 | 0.00053 | 0.008 |
| Iron (Proteinate) 15% | 3.31 | 0.0331 | 0.331 | 5.17 |
| Magnesium (Oxide) 58% | 10 | 0.04 | 0.4 | 6.24 |
| Manganese (Proeinate) 15% | 1.65 | 0.04 | 0.4 | 5.18 |
| Molybdenum (Sodium Molybdate | 0.05 | 0.001 | 0.01 | 0.156 |
| Dihydrate) 39% | | | | |
| Selenium (Sodium Selenite) | 0.00162 | 0.000081 | 0.00081 | 0.156 |
| 44.8% | | | | |
| Zinc (Proteinate) 15% | 50 | 0.04987 | 0.4987 | 7.78 |
| l-Lysine (Mono HCl) | 8.41 | 0.0841 | 0.841 | 13.80 |
| d,l-Methionine | 11.03 | 0.1103 | 1.103 | 17.22 |
| Mixed Tocopherols | | | | 4.68 |
| Choline Chloride | | | | 38.0 |
| Sipernat 50 (Silicon dioxide) | | | | 199.06 |
| Lodex-5 (maltodextrin) | | | | 117.30 |
| Sweet whey | | | | 155.37 |
| BF70 spice | | | | 2.28 |
| Dextrose powder | | | | 250.00 |
| Glucosamine HCl | | | | 100.00 |
| Pernaconniculus-Chondroitin | | | | 200.00 |

*Lactic acid generating bacteria is two-thirds of component and yeast is one-third; lactic acid generating bacteria is 500,000,000 CFU/gm, yeast (e.g., “Saccharamyces”) 250,000,000 CFU/gm

TABLE 5
Stress Formula
(Amounts in mg/lb of body weight unless otherwise stated)

| | | | | Dosage: |
|---|---|---|---|---|
| | | | | mg/ounce |
| Component | High | Low | Preferred | of formula |
| Calcium Pantothenate | 1.80 | 0.09 | 0.028 | 28.00 |
| Vitamin C | 20.00 | 0.056 | 0.017 | 17.00 |
| (ascorbic acid) | | | | |
| Vitamin B₁₂ | 13.00 | 0.13 | 0.198 | 198.59 |
| Vitamin A | 600.00 IU | 0.10 IU | 0.014 | 14.00 |
| Vitamin B₂ | 1.20 | 0.065 | 0.018 | 18.00 |
| Thiamine | 16.00 | 0.0308 | 0.017 | 17.00 |
| Vitamin E | 72.9 IU | 0.729 IU | 0.012 | 12.48 |
| Magnesium Sulfate | 10.00 | 0.113 | 0.113 | 113.00 |
| *Lactobacillus | 10.00 | 0.467 | 1.418 | 1418.00 |
| acidophilus | | | | |
| Sodium Chloride | 166.00 | 0.236 | 2.368 | 2368.00 |
| Dipotassium phosphate | 116.00 | 5.85 | 1.773 | 1773.00 |
| Citric acid | 31.00 | 1.59 | 0.482 | 482.00 |
| Yeast (hydrolyzed) | 180.00 | 0.1957 | 0.283 | 283.00 |
| Glycine | 0.142 | 0.0142 | 0.142 | 141.80 |
| Potassium chloride | 18.00 | 0.93 | 0.283 | 283.00 |
| Vitamin D₃ | 29.00 | 0.729 | 0.002 | 1.56 |
| Dextrose | 40.00 | 2.00 | 21.38 | 21375.00 |
| Artificial flavor | 0.028 | 0.0028 | 28.548 | 28.30 |
| Transfer Factor | 50.00 | 0.05 | 0.75 | 750.00 |
| Sipernat (silicon dioxide) | | | 0.05 | 56.70 |

*10⁹ colony forming units (CFU)/gm

TABLE 6
Performance Formula
(Amounts in mg/lb of body weight unless otherwise stated)

| | | | | Dosage: |
|---|---|---|---|---|
| | | | | mg/oz. |
| Component | High* | Low* | Average* | of formula |
| Super oxide dismutase | 60.0 | 0.6 | 6.0 | 6000.0 |
| Glucosamine salts | 65.0 | 0.65 | 6.5 | 6500.0 |
| Transfer factor¹ (horses, cows) | 15.0 | 0.15 | 1.5 | 1500.0 |
| Transfer factor¹ (goats) | 10.0 | 0.10 | 1.0 | 3000.0 |
| Transfer factor¹ (dogs, cats) | 50.0 | 0.5 | 5.0 | 14000.0 |
| Pernaconniculus-Chondroitin | 16.5 | 0.165 | 1.65 | 1650.0 |
| (mucopolysaccharides) | | | | |
| Boswellic acids | 30 | 0.3 | 3.0 | 3000.0 |
| Di-methyl glycine | 27.0 | 0.27 | 2.7 | 2700.0 |
| Methyl sulfonyl methane | 27.0 | 0.27 | 2.7 | 2700.0 |
| Octocosonol | 2.0 | 0.004 | 0.04 | 400.0 |
| Montmorillinite | 30.0 | 0.3 | 3.0 | 3000.0 |

*These amounts are calculated for livestock animals weighing about 450 to 1,000 pounds, goats weighing about 150 pounds, and dogs and cats weighing from about 8 to about 15 pounds.
¹The amount of transfer factor may vary for different species but the amounts for the other components remain the same for each species.

TABLE 7
Livestock Stress Rumen By-Pass
(Amounts in mg/lb of body weight unless otherwise stated)

| | |
|---|---|
| | Dosage: mg/oz. |
| | (unless otherwise noted) |
| Component | of formula |

Stabilized¹

| | |
|---|---|
| Transfer factor (mammal source) | 3500.0 |
| Transfer factor (avian source) | 1000.0 |
| β-sitosterol (90% phytosterols) | 300.0 |
| Inositol hexaphosphate | 350.0 |
| Olive leaf extracts | 35.0 |
| Aloe extract powder (200:1) | 17.0 |
| Hybridized and non-hybridized | 4000.0 |
| Glucans (from Hybridized Cordycepts | |
| sinensis, Agaricus blazeii, Miatake, | |
| Shitake, Coriolis, Inonotus, Obliquus, and | |
| Poris cocos mushrooms) | |
| Vitamin C | 2000.0 |

Non-Stabilized

| | | |
|---|---|---|
| Vitamin A | 4434 | IU/oz |
| Vitamin D3 | 1140 | IU/oz |
| Vitamin E | 500 | IU/oz |
| Vitamin B1 | 12.77 | |
| Vitamin B2 | 12.77 | |
| Vitamin B12 | 1.5 | |
| Di-potassium phosphate | 1.5 | g/oz |
| Potassium chloride | 207 | |
| Magnesium sulfate | 83 | |
| Calcium pantothenate | 23 | |
| Ascorbic acid | 23 | |
| Lactic acid bacteria | 2.5 × 10⁶ | CFU/oz |
| Yeast (S. cerivisiea) | 15.0 × 10⁶ | CFU/oz |
| Zinc proteinate | 10 | |

*These amounts are calculated for livestock animals weighing about 450 to 1,000 pounds, goats weighing about 150 pounds, and dogs and cats weighing from about 8 to about 15 pounds.
¹Stabilized active ingredients are included in a formulation of 50% soybean oil and 50% active ingredient.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All patents, patent applications, publications, and references cited herein are expressly incorporated by reference in their entirety.

### Example 1

Group I

Two hundred forty crossbred heifers were randomly divided into three groups of 80 calves each. The were individually weighed and received a combination modified-live virus vaccine consisting of infectious bovine rhinotracheitis (IBR) virus, killed bovine viral diarrhea virus (BVD), modified-live bovine respiratory syncytial virus (BRSV) and killed parainfluenza-3 (PI3) virus, a multivalent bacterin-toxoid against 7 clostridial species; a dormectin dewormer (Ivomec); and a progesterone implant. Ten days following processing, the calves were given a booster with the same modified-live vaccine they received initially. One set of 80 calves averaging 440.1 pounds receive a 1 ounce dose of the stress formula, as set forth in column 5, Table 5, dissolved in 1 ounce water via dose syringe at the time of processing. Thereafter, they were given doses of 1 ounce of stress formula daily mixed in the feed (total mixed ration—TMR) for four days after processing. A second set of 80 calves averaging 440 pounds received 1.5 ml/cwt of tilmicosin (Micotil) at the time of initial processing. The third set of 80 averaging 449.9 pounds served as controls. The sets were observed for 26 days after processing at which time each of the calves was again weighed and feed efficiency calculated collectively for each group.

Group II

Two hundred crossbred stocker heifers were randomly divided into four groups of 50 calves each. They were processed in the same manner as the stocks in Group I. One set of 50 calves averaging 441 pounds received 1 ounce of the stress formula as set forth in column 5, Table 4, per day in their TMR for five days. A second set of 50 calves averaging 433 pounds received ½ ounce of the same stress formula in their TMR for five days. A third set of 50 calves averaging 447 pounds received a metaphylactic 1.5 ml of tilmicosin per cwt at the time of initial processing. The fourth set of 50 calves averaging 432 pounds served as controls. Each heifer in all four sets received the modified live virus combination of IBR, PI3, BVD and BSV vaccine booster ten days following initial processing. The groups are observed for 26 days after processing at which time each of the calves were again weighed and feed efficiency was calculated collectively for each group.

A one-way statistical analysis of weight gain of variance was done. F-tests and LSD mean separation was done using alpha=0.05 as type I error rate. Software was SAS (1999), procedure GLM.

Statistical analysis of BRD morbidity utilized: Chi-square analysis with Fisher's exact test with a 0.05 or less probability interpreted as significant to interpret the differences in morbidity rates between groups.

The results are listed in Tables 8 and 9 below.

For Group I, there were no sick pulls (i.e., sick calves for treatment) from the eighty head of heifers that were treated with 1 ounce of stress formula in 1 ounce of water solution via dose syringe the day of processing and 1 ounce of stress formula per day added to the TMR for the four days following processing. There were 17 sick pulls and 4 repulls for BRD from the control group while there were 12 sick pulls and 1 repull from the tilmicosin set.

The heifers in the Group I stress formula set had an average daily gain of 3.63 pounds for the 26 day test period, which is statistically significant when compared to the other two sets. The average daily weight gain (ADG) of the tilmicosin and control sets was 2.96 and 3.08 pounds respectively. Feed efficiency for the stress formula, tilmicosin and control sets was 6.73, 6.94 and 6.66, respectively.

The heifers in the 1 ounce stress formula dosage set in Group II have an average daily gain of 3.2 pounds and those in the one half ounce stress formula dosage set have an average daily gain of 3.05 pounds. The tilmicosin and control sets have an average daily gain of 2.88 pounds and 2.92 pounds, respectively. The feed efficiency for the 1 ounce stress formula is 5.31 while the values for the half ounce stress formula, the tilmicosin and the control sets are 6.09, 6.10 and 5.99. respectively.

There were 11 sick pulls and repulls for treatment of BRD in the set of fifty heifers receiving 1 ounce of stress formula per day added to the total mixed ration for five days, beginning on the day of processing while there were 13 sick pulls and 4 repulls for BRD treatment in the group receiving ½ ounce TF in their TMR for five days. There were 5 sick pulls and 2 repulls from the tilmicosin set during the 30 day test period. Eleven BRD sick pulls and 2 repulls occurred in the control set of heifers.

Upon comparing the differences in the sick pull rate between the sets in Group I, the stress formula appeared to provide significant protection from BRD during the 26 day testing period. Stress formula also significantly increased the average daily gain.

In Group II, the heifers in both sets achieved better weight gain than those in the other two sets. However, in Group II the protection from BRD appears to be less than that of tilmicosin. When one compares the effect of TF on BRD between Group I and Group II, the results appear to be inconsistent until it is realized that the heifers in Group II did not receive their initial dose of stress formula via dose syringe during the processing. This evidence is a strong argument for administration of the initial dose via dose syringe or capsule to assure that every subject receives at least the entire first dose instead of relying totally on receiving the stress formula via the TMR. The heifers that were pulled for treatment in the two stress formula sets may not have eaten a full portion of the TMR on the first critical, stressful day and therefore did not receive enough stress formula to stimulate the immune system.

When comparing the heifers receiving the full ounce per day stress formula with the set receiving a half ounce per day, there is not significant differences in the performance of the heifers. It is very possible that if both dosages are administered initially via dose syringe or capsule the differences may be even less.

It should be noted here that the value of the weight gained by the stress formula sets in excess of the weight gained by the other sets in Group II was more than enough to compensate for the cost of treatments for BRD in the stress formula sets.

In high risk cattle that are not preconditioned such as the heifers in these studies, direct stimulation of the immune system with stress formula along with vaccine administration appeared to indeed enhance the level of immunity against BRD. Stress formula appeared to decrease the need for antibiotic treatment and or enhance the effectiveness of antibiotic therapy.

TABLE 8
Results for Group I
1 oz. stress formula daily - drenching the first day followed by
4 days of top dressing

| Treatment | # of | | | | | Feed | Sick |
|---|---|---|---|---|---|---|---|
| Group | heifers | ADG | Kg (lbs) | Pulls | Repulls | Efficiency | pulls |
| Stress | 80 | 3.63 | 200.0 | 0 | 0 | 6.73 | 1.65 |
| Formula | | | (440.1) | | | | |
| (1 oz/day) | | | | | | | |
| Tilmicosin | 80 | 2.96 | 200.0 | 12 | 1 | 6.94 | 1.35 |
| (Micotil | | | (440.0) | | | | |
| 1.5 ml/ | | | | | | | |
| cwt) | | | | | | | |
| Control | 80 | 3.08 | 204.5 | 17 | 4 | 6.66 | 1.40 |
| | | | (449.9) | | | | |

TABLE 9
Results for Group II
Stress Formula daily - 5 days of top dressing only

| Treatment | # of | | | | | Feed | Sick |
|---|---|---|---|---|---|---|---|
| Group | heifers | ADG | Kg (lbs) | Pulls | Repulls | Efficiency | pulls |
| Stress | 50 | 3.20 | 200.5 | 11 | 4 | 5.31 | 1.45 |
| Formula | | | (441.0) | | | | |
| (1 oz/day) | | | | | | | |
| Stress | 50 | 3.05 | 198.8 | 13 | 4 | 6.09 | 1.39 |
| Formula | | | (433.0) | | | | |
| (½ oz/ | | | | | | | |
| day) | | | | | | | |
| Tilmicosin | 50 | 2.88 | 203.2 | 5 | 2 | 6.10 | 1.31 |
| (Micotil | | | (447.0) | | | | |
| 1.5 ml/ | | | | | | | |
| cwt) | | | | | | | |
| Control | 50 | 2.92 | 196.4 | 11 | 2 | 5.99 | 1.33 |
| | | | (432.0) | | | | |

### Example 2

A herd of cattle in Fort Bidwell, Calif. had a chronic problem with calf dysentery with a death rate of 63% and morbidity of 90%. This problem had persisted for seven years. Treatments that resulted in no improvement included the antibiotics tetracycline, mycotil, sulfur and penicillium along with the other traditional treatments such as fluids and anti-diarrheal medications like kaopectate. The University of Califronia, Davis, and the University of Washington were unable to provide a solution. Forty test calves weighing around 100 pounds each were treated daily with one ounce of stress formula as shown in column 5, Table 5 delivered in a gelatin capsule for two days and 60 calves acting as controls received nothing for prophylaxis. In the test calves one animal died because it had been medicated too late but none of the other test animals exhibited any symptoms of disease. However, the control calves had a 90 percent rate of dysentery which was the same as in previous years. The calves were treated with stress formula immediately after they broke with the dysentery and they cleared up. The new calves in the herd are now being treated with one ounce of stress formula as shown in column 5, Table 5 in gelatin capsules and they showed the same results with one gel cap daily for two days as the test calves. The last twenty calves in the herd that have been treated with the stress formula protocol have been turned out to pasture and are 7% heavier and have better coats and attitude than the test calves. Neighboring ranchers with calves having similar dysentery problems have also started testing the stress formula protocol and have obtained similar successful results.

### Example 3

A farm in Pennsylvania had 40 ovum donor cows that were losing all their calves and some of the adult cows also appeared ill. The University of Ohio diagnosed the cows and calves as suffering from Clostridium Perfrengens type A. The cows and calves were first treated with several available antibiotics with no success. The morbidity rate for the calves was 100% and mortality was 80%. A protocol was begun of treating calves weighing about 80-100 pounds each with one ounce daily of the stress formula as shown in column 5, Table 5 for seven days when they were born. These calves were given no antibiotics. Since the initiation of this protocol approximately 30 calves have been treated, no dysentery has been observed in the herd and no more calves have died.

### Example 4

A herd of 130 head of cows and calves in Columbus Nebraska was suffering from chronic dysentery of coliform origin. Approximately 60% of the calves appeared affected. Treatment with antibiotics and fluids provided moderate success with an approximate ten percent mortality rate. Ten of the calves weighing about 80-100 pounds each and suffering from the dysentery were then treated daily with one ounce of the stress formula as shown in column 5, Table 5 for three days. After the three days on the protocol the 10 calves no longer exhibited signs of dysentery. However, the untreated calves still had dysentery problems.

### Example 5

Over fifty cases of benign tumors in cats (2.2 gm/daily as shown in column 5, Table 4), dogs (28.37 gm/daily as shown in column 5, Table 3) and horses and cattle (5 oz./daily as shown in column 5, Table 2) have been treated with the premix formulations. These tumors range from benign sarcoids, to pappilomas. In general, the tumors have been reduced from 40% to 80% and and even completely in some cases. Malignant tumors such as oral squamous cell carcinomas have been reduced in dogs receiving 28.37 gm/daily of the premix formula as shown in column 5 of Table 3 and in cats receiving 2.2 gm/daily of the premix formula as shown in column 5 of Table 4.

### Example 6

One hundred head of cattle weighing 450 pounds arrived in the feedlot from a two-hour truck ride from a ranch and are just weaned off the cows. Fifty of the cattle vaccinated are processed with routine vaccination and worming and one injection of Micotil and act a controls. The other fifty cattle are vaccinated, wormed and each given one ounce of solution containing 1500 mg transfer factor and 1418 mg of lactic acid producing bacteria as shown in Table 5. This dose is given orally to each of the test cattle for four more days. After 30 days on the transfer factor and lactic acid producing bacteria, the test cattle are each 10 pounds heavier than the Micotil cattle.

### Example 7

One Hundred head of cows calving are having a serious outbreak of Clostridium Perfrengens type A with a calve morbidity rate of 80% and a mortality rate of 30% given traditional treatment. The calves weighing about 110 pounds each are given 750 mg of transfer factor and 1418 mg of lactobacillus acidophilus (109 colony forming units (CFU)/gm) for two consecutive days and the incidence of clostridium is reduced to 20% with mortality reduced to 5%.

### Example 8

Five hundred head of stockers enters the feed lot weighing about 600 pounds each after a 6-hour trailer ride from the ranch and are immediately processed (i.e., wormed and vaccinated). Two hundred fifty head or every other calf is given 750 mg transfer factor, 283 mg yeast, and 2368 mg lactic acid according to Table 5. The other calves are processed and some are given Micotil and others are given Liquarnycin and sulfas to test different products at recommended doses.

After 40 days, the transfer factor, yeast, and lactic acid bacteria calves are 12 pounds heavier than the other calves and morbidity is 30% less in the transfer factor, etc., calves than in the other calves. Carcass yield data shows major improvement on the transfer factor cattle with large ribeye, less carcass waste, and higher yield.

### Example 9

A small dairy herd of 100 cows has Clostridium Perfrengens type A chronic dysentery in its first born calves. Calves are being lost with conventional treatment. The remaining calves are treated with formula a of 1300 mg transfer factor and 1418 mg lactic acid producing bacteria and 283 mg yeast as shown in Table 5 daily for 5 days after birth, mixing the product into solution and drenching each calf. Morbidity is reduced 60% and mortality reduced 80%.

### Example 10

This example compares oral dosing of bovine transfer factor with metaphylactic antibiotic (Micotil) treatment of calves and their effects on performance and health of stressed feeder cattle.

Approximately 600-700 feeder calves (400 to 500 lb each) were placed into large pens and offered ad libitum access to clean water and long-stem hay prior to processing. Within 24 hours after arrival, weight and rectal temperatures were recorded for each animal. Cattle were worked through the processing facility at random, and uniquely identified with numbered ear tags. Each animal was treated for internal and external parasites (Phonectin) and vaccinated against common viral (Bovishield 4) and clostridial (Fortress-7) diseases.

Each load of calves were sorted four ways into groups 23-28 head each. Every other animal received a 1-ounce oral dose of non-encapsulated bovine transfer factor as set forth in Table 5 (administered as an oral liquid drench), and the remaining animals received 1.5 ml/100 lb BW of Micotil. Animals assigned to the Bovine Transfer Factor group were supplemented with bovine transfer factor at 1 ounce per head daily as a ration top-dress on days 2, 3, 4 and 5. Groups were assigned randomly to consecutively numbered pens. Cattle were re-vaccinated using a 4-way viral vaccine (Bovishield-4) on day 7 after initial processing and were temperature recorded.

Experimental diets provided approximately 45% roughage and 55% concentrate. The amount of feed offered to each pen of cattle were determined at approximately 0700 h each morning. Cattle were fed amounts sufficient to result in only traces of unconsumed feed in the bunk the following morning. The entire daily ration for each pen was delivered at approximately 0800 h every day. Residual feed, when in excess, were removed from the bunk to prevent spoilage. Feed removed was weighed and accounted for in subsequent calculations of feed consumption.

Animals were monitored daily for clinical signs of respiratory disease. Cattle that exhibit clinical signs of respiratory disease, including depression, lethargy, anorexia, coughing, rapid breathing, nasal and/or ocular discharge were identified as candidates for therapeutic treatment. Animals were assigned a clinical score ranging from 1 to 4. A clinical sore of 1 is used to identify mild respiratory disease, a clinical score of 2 indicates moderate disease, a score of 3 indicates severe respiratory diseases, and a clinical score of 4 represents a moribund animal. Animals assigned a clinical score of 1 or greater were removed from their pen (pulled) and taken to the processing area for determination of body weight and rectal temperature. Animals with a clinical score of one or greater received antibiotic therapy.

All animals that were treated received the standard protocol for respiratory disease, which includes subcutaneous injection of tilmicosin (Micotil®) at a dosage of 10 mg/kg. Rectal temperature was recorded, and cattle were returned to their original pen following treatment. Where necessary, treatment was repeated after 48 hours. Information pertaining to morbidity, mortality, rate of gain and feed intake was collected throughout the experiment.

At the end of the receiving phase, cattle were individually weighted and a 10-ml aliquot of blood retained for recovery of plasma. Receiving pens were consolidated to provide equal distribution of cattle from each treatment into each of two pastures. Cattle were then transported for summer grazing on native grass pastures. Upon completion of the grazing phase, cattle were gathered from pastures and transported for finishing. Cattle were distributed among four feedlot pens, with cattle from 6 pens consolidated into a single feedlot pen (approximately 150-180 head).

The results from this experiment are set forth in Table 10. As can be seen, these animals receiving the transfer factor treatment had significantly higher pulls for antibiotic treatment as compared to animals treated with Micotil, i.e., 73% versus 48% for first time treatment, 32% versus 14% for second time treatments and 17% versus 50% for third time treatments.

These results indicate that transfer factor did not work as well as Micotil when used to treat a stressed population of cattle.

TABLE 10

| Item | Micotil | Transfer Factor |
|---|---|---|
| No. Head | 333 | 332 |
| Initial weight, lb | 492.1 | 495.6 |
| Initial rectal temperature, deg F. | 102.5 | 102.6 |
| 7-day weight, lb | 502.2 | 506.3 |
| 7-day rectal temperature, deg F. | 102.4 | 102.3 |
| Dry matter intake, lb | | |
| Last 7 days | 12.8 | 12.1 |
| Last 21 days | 9.8 | 9.6 |
| 1^{st}-time treatments, % of total | 48.05 | 73.49 |
| Retreatments, % of total | 14.11 | 31.93 |
| 3^{rd}-time treatments, % of total | 4.50 | 17.47 |
| Deads, % of total | 0.60 | 0.30 |

### Example 11

In Vitro Protein Degradation. In vitro incubations of rumen fluid alone (control), with casein, or with TF were conducted. Rumen contents were obtained from two ruminally cannulated Jersey steers fed a diet containing 76% steam-flaked corn, 10% alfalfa hay, 3% soybean meal, 1.2% urea, 5% cane molasses and 4.8% of a mineral vitamin premix (DM basis) offered for ad libitum consumption. Whole rumen contents were strained through two layers of cheesecloth and the removal of any particle-associated organisms was attempted by washing solid residue remaining on the cheesecloth four times with prepared McDougall's buffer using a total volume equal to that of the original volume of strained rumen fluid. The strained rumen fluid and buffer solution mixture was then filtered through eight layers of cheesecloth and composited.

The final inoculum contained (per liter) 450 mL of strained rumen fluid, 450 mL of buffer extract from washed solids, 234 mg of 2-Mercaptoethanol, 50 L of a maltose solution containing 100 mg/mL of maltose, 25 mL of a 60 mM hydrazine sulfate solution and 25 mL of a chloramphenicol solution containing 1.80 mg/mL of chloramphenicol. Hydrazine sulfate and chloramphenicol were added in an attempt to inhibit microbial uptake and metabolism of NH₃ and AA.

Forty mg of N from either casein or Stress Formula (N concentrations of casein and Stress Formula were predetermined according to analysis of Kjeldahl N¹⁶) were weighted into 500 mL Erlenmeyer flasks and 100 mL of McDougall's buffer was added. Flasks containing buffer alone (control), buffer plus casein, or buffer plus Stress Formula were then incubated for 1 hour at 39° C. in a temperature-controlled room. A total of 12 flasks were used, providing four replications per treatment.

In vitro incubations were initiated by adding 200 mL of inoculum to each flask while flushing with CO₂. The incubation was 4 hour in duration and a 1-mL sample was collected immediately following the addition of inoculum (0 hour) and every 30 minutes thereafter. Upon sampling, the 1-mL samples were placed into disposable microcentrifuge tubes containing 0.25 mL of chilled 25% w/v trichloroacetic acid and stored at −20° C. until subsequent analysis.

Upon analysis, samples were thawed at room temperature and then centrifuged for 15 minutes at 21,000×g and the resulting supernatant was analyzed for NH₃ and total amino acid concentration according to Broderick and Kang¹⁷ using a Technicon III AutoAnalyzer^{f}.

Calculation of Protein Degradability Rate. Although in vitro incubation was conducted over the course of 4 hours, NH₃ and total amino acid concentrations increased only through 1.5 hours, after which NH₃ and total AA concentration began to decrease, suggesting uptake of NH₃ and total amino acid by microbes. Therefore, only time points between hours 0 and 1.5 were used in calculating rate of in vitro protein degradation. In vitro protein degradation at each time point was calculated using the formula: Percent protein degraded=blank corrected ([NH₃—N])+([total amino acid−N])/mg N added to flasks. Percent undegraded protein at each time point was calculated using the formula: 100−percent undegraded protein.

Statistical Analysis. Rate of protein degradation was determined using regression analysis to regress the natural logarithms of percent-undegraded protein against time. The resulting slopes represented the rate of protein degradation in fraction/hour. Slopes representing the rate of protein degradation were analyzed using ANOVA^{g}, with flask serving as the experimental unit and model effects consisting of protein source.

### Example 12

A cattle feedlot operation having 3,800 head of feeder cattle participated in a study using the composition detailed in Table 7. Typical practice for much of the industry is to purchase feeder cattle from ranches or sale barns and then have the cattle transported to a feedlot. Upon arrival, animals typically weigh 350 to 550-lbs. Cattle are treated, fed and finished to market weight. The feedlot participating in the study has employed the following treatment protocols over several years: Micotil® administered at 1.5 cc per cwt; TSV-2 (intranasal IBR-PI-3); Triangle 4 (IBR-PI-3, BVD, BRSV, Pasturella hemolyticum and Haemophilus somis); Ivermectin (pour-on); Aureomycin at a rate of 80 mg daily for 21-days, in chopped, mixed grass including trace mineral salts. In the year preceding the study, the above protocols resulted in mortality of 15 head (3.9%), morbidity of 1140 head (30%), and chronic pulmonary illness (lungers) of 200 head (5.3%). The participating feedlots protocols result in statistics similar to, or better than, national averages for 3,800 head of cattle, which would have a mortality rate of 247 head (6.4%) and a morbidity rate of 25%-35%.

During this study the participating feedlot's standard protocols were supplemented with the composition detailed in Table 7. Supplementation of the protocols included three consecutive treatments each comprising a single oral administration of a 1 oz. gel cap on day one followed by 1 oz. administrations of the formulation top-dressed for two consecutive days.

The results of the study reflect a dramatic and exceptional improvement over the previous year, as well as the national averages, by adding the composition detailed in Table 7 to the prior protocols. In particular, mortality rates dropped 90% to 15 head (0.39%), morbidity rates dropped 68% to 342 head (9%), and chronic pulmonary illness dropped 84% to 32 head (0.84%).

In addition to the improved mortality and morbidity outcomes, the study also reflected that the addition of the composition detailed in Table 7 to the prior protocols resulted in a significant increase in weight gain. Under the prior treatment protocol, the average weight gain was 45 pounds in the first 30 days. Under the supplemented protocol, the average weight gain was 80 pounds in the first 30 days.

### Example 13

The constant and ongoing battle to maintain acceptable Bulk Tank Somatic Cell Counts (BTSCC) represents one of the single largest financial drains to the dairy industry. Individual cost per cow treatment can run in excess of $250. Recent studies state that 34.5% of all dairy cows have SSC in the 200,000 to 229,000 range. Growing pressure to reduce antibiotic use, emergence of resistant microbial strains, and the recent upward trend in national BTSCC, further demonstrate the serious nature of this problem, and the growing need to lower and maintain reduced Somatic Cell Counts. Financial rewards in the form of quality premiums add additional importance to SCC control. Accordingly, a study was undertaken to determine if the composition detailed in Table 7 could be used to efficiently lower BTSCC.

The study included 26 cows selected for their high somatic cell counts. The Control Group (13 cows) had a beginning average SCC of 1,854,811. The Treated Group (13 cows) had a beginning average SCC of 2,374,000. Cows in the control group received standard protocols during the 60-day study period. Treated cows received 1 oz. of the composition detailed in Table 7 daily for three consecutive days followed by three days off for three cycles (a total of nine treatments).

SCC testing of the Control and Treated Groups 26 days later revealed that the Control Group had an SCC of 2,049,636 (an increase of 10.5%), while the Treated Group had an SCC of 957,455 (a decrease of 59.7%). Accordingly, the Treated Group had a 70.2% improvement over the Control Group. Furthermore, SCC counts at 90-day testing indicated a 26% reduction in SCC demonstrating a residual effect of the composition.

### Example 14

64 high stress stockers were purchased and 32 (Treated Group) were initially administered two 1 oz. gel caps containing the composition detailed in Table 7, while the remaining 32 (Control Group) were left untreated. The Treated Group were also given 1 oz. daily of the composition for an additional two days. Neither the Treated Group or the Control Group received antibiotic treatment. After three weeks, 5 calves from the Treated group required treatment for morbidity while 12 from the Control Group required such treatment (a 60% improvement in morbidity reduction). In addition, while 1 calf died in the Control Group, no calves died in the Study Group.

### Example 15

Seven goats each having severe pinkeye, Chlymidia, other bacterial infections or were going blind. All seven were on standard medications for three weeks with little or no improvement. All diseased goats were then administered 1 oz. daily of the composition detailed in Table 7 for 14 days. Two goats breaking with disease stopped progress in about 48 hours, the other goats returned to normal in 10 days with no scarring of the eye, and warts also dropped off the infected goats. No antibiotics were used in the protocol.

### Example 16

### Growth of the Fungi Cordyceps

The ideal medium for solid substrate growth of Cordyceps is as follows: 1 part white proso millet (husk on) to 4 parts of white Milo (husk on) with the addition of 0.8% w/w of ground oyster shell and 1% w/w vegetable oil (peanut oil or soybean oil). Add water to equal 50% total moisture in the sterilized substrate. Precooking the grain mixture for 4-6 hours prior to sterilization tends to trigger a much faster growth response from the Cordyceps. On this medium, Cordyceps can be grown for long periods of time, allowing nearly complete conversion of the substrate to mycelium and the full expression of secondary metabolites from the Cordyceps. The resultant Cordyceps when grown on this substrate is about 3-4% residual grain, or about 96-98% pure mycelium. The real benefit to this method of growing is the capture of the entire compliment of extra-cellular metabolites produced throughout the entire growth process. With the addition of certain growth triggering compounds to this mixture, Cordyceps sinesis is easily induced to fruit in culture without any insect material being present. However the formation of the fruitbody on this medium does not result in any significant change to the analytical chemistry profile.

Using the above described substrate, the complete chemical profile of the cultivated Cordyceps still does not approach that of the wild collected Cordyceps unless it is grown under very specific conditions. Cordyceps sinensis produce a relatively large amount of free Adenosine when grown at normal atmospheric oxygen levels and room temperatures. It will also produce a large quantity of Uridine and Guanosine. But there is very little if any Cordycepin produced, and virtually no Hydroxyethyl Adenosine. For the organism to produce these compounds, it needs to be growth-stressed through the absence of oxygen, a drop in temperature and the total absence of light. Just growing it under cold and anaerobic conditions from the start does not work, since when Cordyceps is grown under those conditions it forms a yeast-like anamorph that has a very different chemical profile. It must first be grown hot and fast, then tricked into converting its “summertime” metabolites into target medicinal compounds. To get these target compounds, a strict growth protocol was followed. After inoculation on to the milled/milo substrate, the Cordyceps is grown at 20-22° C., in diffuse light and at sea level atmospheric oxygen for 28-30 days. It is then moved into a controlled environmental chamber, where the oxygen is dropped to 50% atmospheric oxygen, i.e., approximately 10% oxygen. The remainder of the growth atmosphere is made up of nitrogen, carbon monoxide and carbon dioxide. The temperature is lowered to 3° C., and all light is excluded. It is held under these conditions for about 15-20 weeks. This results in much of the Adenosine being converted to Cordycepin, Dideoxy-adenosine and Hydroxyethyl-adenosine. Many other unique nucleosides are also produced, with a final chemical profile identically matching that of the wild Cordyceps.

### Example 17

### Hybrid Glucan Formulation

Once the substrate and growth parameters were determined to optimize the target compounds, the chemical profile differences from different strains of Cordyceps sinensis was determined. Since there are so many strains of Cordyceps, and each strain has its own unique chemical profile, all of the strains obtained were tested. None of the known strains was shown to produce nearly the quantities of active ingredients found in the wild Cordyceps. In order to quantitatively increase the target compound production hybridization experiments of Cordyceps strains were carried out; to cross breed them in order to gain greater production of target compounds. Various experiments were conducted to get different strains of the fungi to perform their own nuclear fusion. Nicotinic acid for instance, can be used to create hybridized mycelium. This compound is difficult to use and yields unreliable results. After trying several different compounds to trigger this fusion, it was discovered that snake venom worked best.

Snake venom was purified from the Western Diamondback Rattlesnake (Crotalus atrox), [Sigma Scientific, St. Louis, Mo., USA] for hybridization experiments. The snake venom is added to the agar medium in quantities that alters the growth but does not prove toxic to the strain in question. This range of snake venom is from 10 mg to 30 mg per 300 ml of agar medium. The venom is not heat stable and must be added aseptically after sterilization of the medium. The agar used for this hybridization in an Aloha Medicinals, Inc., Maui, Hi., proprietary agar named R7 Agar, consisting of malt extract, activated carbon, minerals and humus—the carbon-rich ash residue from a coal burning industrial process. The exact formulation is set forth in Table 11. Other agars can be used as well.

TABLE 11
Snake Venom/R7 Agar Recipe

| | | |
|---|---|---|
| 2.1 | L | Distilled Water |
| 50 | g | Light Malt Extract |
| 34 | g | Agar |
| 10 | g | Humus |
| 5 | g | Activated carbon |
| 1 | g | MgSO₄ |
| 10 | ml | 1% KOH solution |

| | |
|---|---|
| As Required | C. atrox venom |

Petri dishes of this R7 agar medium are inoculated with mycelium from two different strains of the Cordyceps genus. These are usually two varieties of C. sinensis, although we have also crossbred C. sinensis with other Cordyceps species such as C. militaries, C. sobolifera and C. ophioglosoides. These different strains when inoculated together onto one petri dish will normally grow towards each other until they almost meet, at which point they form a zone of inhibition, where neither strain can grow. Eventually, one strain may prove stronger than the other and overgrown the plate, but they will remain genetically distinct; two different cultures residing in the same petri dish.

With the addition of a sufficient of snake venom to the agar, the two cultures grow towards each other until they meet and form their mutual zone of inhibition. This period of inhibition is short lived, however, for in only about 2 or 3 hours, the colonies each start sending out mycelial strands into the zone of inhibition. These strands grow together and exchange nuclear material through their venom-weakened cell walls. They form a hybrid strain at this point of mutual contact of new hybrid strain that is distinctly different from either of the parent strains. Within about 4 hours after first forming the zone of inhibition, the hybridization is complete and the colonies resume rapid growth towards each other. They become three colonies, the original two and a new hybrid strain.

A section of the newly formed hybrid is carefully removed from the original zone of inhibition at the precise time that the colonies begin to fuse. That is, during hour 3-4 after the initial meeting of the colonies. The hybrid is transferred to a new petri dish containing normal (non-snake venom) Agar. One method of determining hybridization is to inoculate a new dish containing normal agar with all three strains, the original two and the suspected hybrid. If the hybridization has in fact taken place, these are now three distinct colonies, and will form a mutual three-way zone of inhibition. If hybridization has failed to occur, then the suspected hybrid will readily fuse with each other or the other of the original colonies, proving that the suspected hybrid will readily fuse with either one or the other of the original colonies, proving that the suspected hybrid is not genetically distinct from the original.

Once a hybrid is confirmed, it is tested for growth parameters. If it appears to be a vigorous and hardy grower on the substrate, it is grown out of a quantity of mycelium, harvested and analyzed for active ingredients. Through repeated testing in this way, hybrid strains are made that are easily grown in solid substrate culture, with a potency greater than any other cultivated strain and at least equal in potency to the highest quality wild Cordyceps. This new strain is Cordyceps sinensis Alohaenis.

### Example 18

### Treatment of Stressed Cattle

The transfer factor formulation set forth in Table 7 was used to study live stock under stress. This rumen by-pass formulation was administered to calves in the amount of 1 ounce per head per day for 4 days. There were 318 head of calves that were treated with the transfer factor formulation. There were 180 head of calves in the control of population. All calves were vaccinated and warmed.

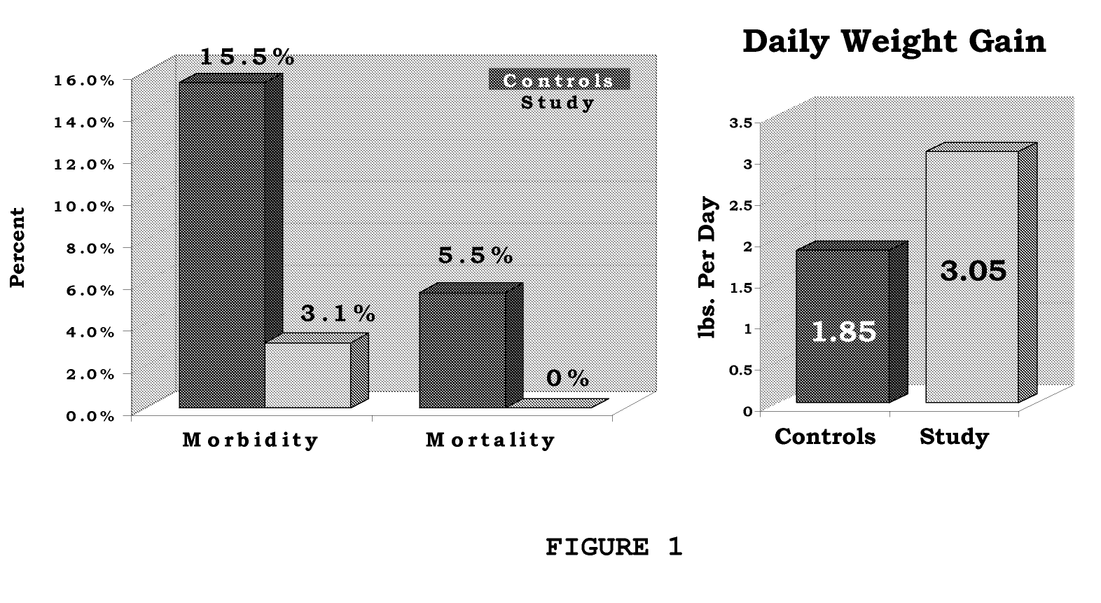

The results from this experiment are found in FIG. 1. As can be seen, the morbidity in the control population was approximately 15.5% whereas the morbidity in the transfer factor treated population was 3.1%. In addition, the mortality in the control population was 5.5% whereas the mortality in the transfer factor treated population was 0%. The daily weight gain for the controls was 1.85 pounds per day whereas the population treated with transfer factor had a daily weight of approximately 3.05 pounds per day.

### Example 19

In another study, 585 calves were treated for 3 days with 1 ounce of the transfer factor formulation of Table 7 each day and 1 ounce of the formulation of Table 7 during re-vaccination on day 12. A control population of 29 calves did not receive the formulation of Table 7. All calves in the study received vaccines and antibiotics (Micotil or A-1A) and wormer (Ibomec). The calves were conditioned for 4-6 days to 45 days, dehorned if necessary, and all bulls were castrated. Average daily weight gain was calculated based on the in and out weights at the conditioning yard.

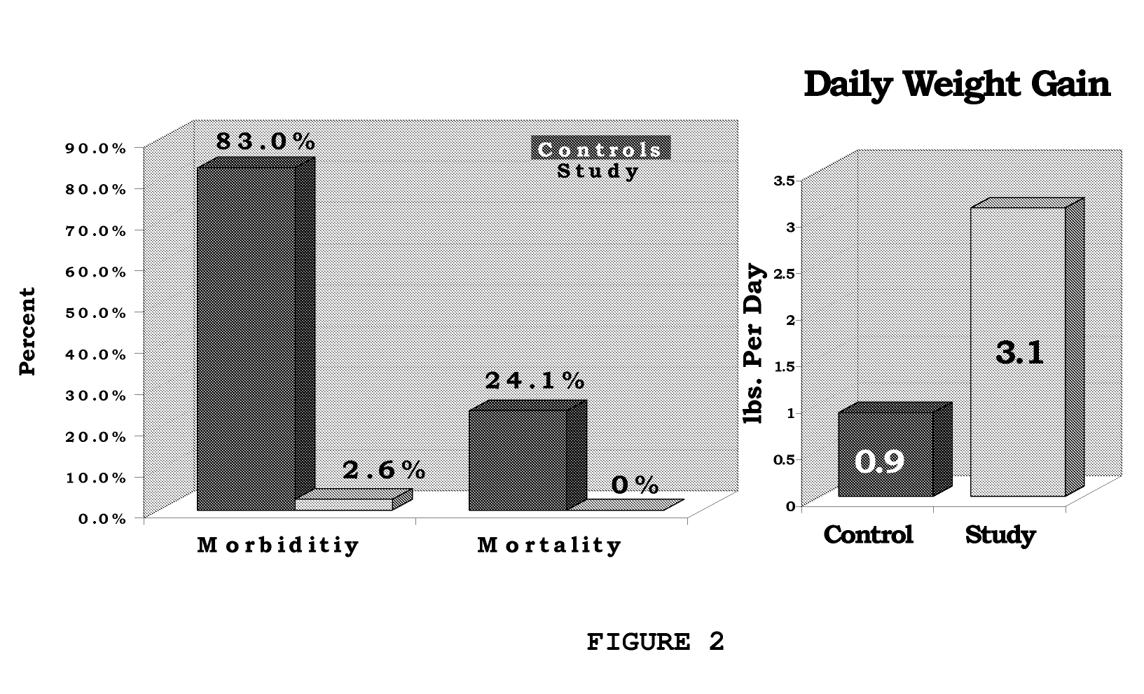

As can be seen in FIG. 2, the morbidity of the control group constituted 83% whereas the morbidity in the transfer factor treated population was only 2.6%. Similarly, the mortality rate in the control population was 24.1% versus 0% in the population treated with transfer factor. In each case, the deaths in the control population were the result of bovine respiratory disease. In addition, the daily weight gain in the control group was less than 1 pound per day whereas those treated with transfer factor gained approximately 3.1 pounds per day.

### Example 20

Twelve (12) high stress salebarn calves that were directly weaned off their mother were used for this study. All needed further processing, e.g., vaccination and worming. Males were also castrated. Of the 12 calves, the four bulls were castrated at the barn, transported to a farm and processed. At 6 PM the first blood was drawn for this study. Blood samples were taken each 12 hours for three days. Thereafter, the AM and PM blood samples were taken on day 5-7-9-12 for a total of 168 blood samples. They were analyzed for cortisol, thyroid function and insulin.

The calves were randomly gate cut into two groups of six. Three bulls were castrated in the control group. The study population also had a very light calf weighing 355 pounds. The lightest calf in the control group was 455 pounds. Study calves were given two gel caps of transfer factor livestock stress rumen bypass formula equivalent to one ounce for 3 days then day 12, one more ounce. The control calves were given a placebo of wheat bran in the same size gel caps. All calves were processed at the same time and the diet was the same for both groups.

The base line cortisol average for the study calves for AM and PM blood samples was 11.98 ng/ml on the low side and 21.41 ng/ml on the high side. This is a difference of 44%. The base line cortisol average for the controls for AM and PM blood samples was 17.6 ng/ml on the low side and 40.18 ng/ml on the high side. This is a 56% difference.

The difference in cortisol levels in the study group in the first 12 hours with the study group receiving product in the first 12 hours prior to this blood draw was 22.56% for the study group and 46.4% for the controls. This is a 109% difference indicating a dramatic adjustment in the first 12 hours of stress with calves receiving product. One would expect the highest degree of stress on day 5. The overall average difference in AM and PM cortisol levels was 34.9% for the study group and 67.63% for the control group. This is a 94% difference.

Diurnal rhythm for average of the total study was (1) study calves 44.50%, (2) controls 32.35%. This is a 27.15% difference. The largest difference in rhythm was at day 7, with study calves being 38.01 to controls 11.21 or a 70.52% difference.

The calculation of diurnal rhythm eliminated the first and last blood draw of the first 3 days to give an actual AM, PM readings. This showed little difference in total average values. The advantage to study calves was 26.87% better rhythm when the first draw and last draw of the first 3 days was eliminated.

The average thyroid data for the study calves was 59.40 ng/ml of T4. The average for the controls was 54.30 ng/ml of T4 or a 8.9% difference. The biggest difference was on day 5, with study calves being 59.50 ng/ml of T4 and 47.05 ng/ml for controls. This is a 21% difference. Higher levels of T4 were observed on six of the seven blood samples from the study calves.

The insulin average for the study calves was 57.36% and the control calves was 19.37%.

The steady increase of insulin on the study calves from 18.95 IU/ml on day 1 to 57.36 IU/ml on day 12 indicates an animal with increasing feed consumption. The control calves had insulin levels on day 1 of 12.47 IU/ml to on day 12 of 19.37 IU/ml indicating poor feed consumption. The study calves had a 35.41% increase in weight over 60 days. The control calves had a 26.84% increase in weight gain over the same time period. There was a 24.20% advantage in weight gain for the study calves. The higher insulin calves appeared to have the fastest rate of weight gain.

A summary for individual daily totals for AM and PM blood samples for each category is as follows:

(1) Sixty-eight (68) out of 84 blood samples from the study calves had higher insulin levels than controls, indicating more feed consumption for the study calves.

(2) Six (6) out of the 7 days, the average thyroid function for the study group was higher than the controls, indicating less stress on the study calves.

(3) Out of 84 daily cortisol averages for AM and PM blood samples, all study calves without exception had lower cortisol levels than the control group.

(4) Out of 84 rhythm studies of AM and PM blood samples, 71 of the blood samples from the study calves had better rhythm as opposed to 13 of the samples of the control calves that had better rhythm.

(5) The total weight gain over the first 60 days:
(a) Study group—1585 pounds.
(b) Control group—1200 pounds.

APPENDIX 1
HUMAN AND BOVINE PATHOGENS:
POTENTIONAL CROSS REACTIVITY

| Human Pathogen or Disease | Commonality | Bovine Pathogen |
|---|---|---|
| BACTERIA | | |
| Travelers Disease (E. coli) | very | Toxigenic E. coli |
| | very | Campylobacter jejuni |
| Bloody diarrhea/hemolytic uremia | increasing | E. coli 0157:H7 Verotoxic |
| Salmonellosis/Typhoid Fever | common | Salmonella thyphimurium, |
| Salmonella typhosa | | dublin |
| Diarrhea, from food or water | very | Campylobacter jejuni |
| Clostridial Infection (non-tetanus) | common | Clostridia (many species) |
| C. dificil | | |
| Mycobacterium Infections | | Mycobacterium species |
| johnei, Crohn's Disease | common | common in Jersey cattle |
| Staphylococcal super infections | common | Staph. aureus |
| Streptococcal infections | common | Streptococcus |
| Endocarditis | common | Beta Strep. |
| Superinfection | increasing | S. pyogenes |
| S. pyogenes | increasing | |
| Enterococci | common | Enterococci (most spp. & VRE) |
| Hospital/VRE strains serious | common | |
| Helicobacter pylon (ulcers) | common | Bovine/Porcine association |
| VIRUS | | |
| Influenza | common | Influenza virus |
| Pneumonia Resp. Syncytial Virus | common | Bovine Resp. Sync. Virus |
| Papilloma, Condylomaya | common | Bovine Papilloma Virus |
| Virus Diarrhea | common | Bovine Virus Diarrhea |
| Rotavirus | | Rotavirus |
| | | Coronavirus |
| Cytomegalovirus | common | Bovine CMV and IBR |
| Herpes Infections | common | Bovine Rhinotracheitis |
| HIV (Retrovirus) | common | Bovine Immune Deficiency Virus |
| Rhinovirus (common cold) | very | Bovine Rhinovirus |
| YEAST, FUNGI and PROTOZOA | | |
| Candidiasis | common | Candida exp. common |
| Cryptosporidiosis | very | Calf diarrhea, C. parvum |
| Giardiasis | common | Calf diarrhea, G. lamblia |
| OTHER | | |
| Mycoplasma pneumonia, arthritis | common | Bvn. Mycopl. Pneumonia |

APPENDIX 2
HUMAN AND AVIAN PATHOGENS:
POTENTIAL CROSS REACTIVITY

| Human Pathogen | | |
|---|---|---|
| or Disease | Commonality | Avian Pathogen |
| BACTERIA | | |
| Travelers Diarrhea (E. coli) | very | Toxigenic E. coli |
| | very | Campylobacter jejuni |
| Bloody diarrhea/hemolytic | increasing | E. coli 0157:H7 verotoxic |
| uremia | | |
| Diarrhea | | O1, O2, O47, others |
| Salmonellosis | very | Salmonella sp. |
| Diarrhea, from food and | very | Campylobacter jejuni |
| water | | |
| Clostridial Infection | common | Clostridia sp. |
| Pasteurellosis | very | Pasteurella multocida |
| Pneumonia | common | Haemophilus gallinarium |
| | common | Mycoplasma gallispeticum |
| | common | Chlamydia pneumona |
| Systemic infection | common | Erysipeloxthrix insidiosa |
| Diarrhea, systemic infection | very | Lisreria monocytogenes |
| VIRUS | | |
| Chicken pox | very | Fowl pox |
| Influenza | very | Influenza virus |
| Infectious bronchitis | common | Infectious Bronchitis |
| Adult Leukemia virus | rare | Marek's disease virus |
| (ATLV-1) | | |
| Pneumonia | common | Paramyxovirus |
| Herpetic infections | common | Herpes simplex virus |
| FUNGAL | | |
| Pneumonia, systemic | very | Aspergillus sp. |
| disease | | |
| Diarrhea, systemic disease | very | Aspergillus sp. |
| Diarrhea, thrush, vaginitis | very | Candida albicans |
| Systemic disease | very | Histoplasma capsulatum |
| Systemic disease | very | Coccidia |
| PARASITES | | |
| Trichomoniasis | very | Trichomonas |
| Diarrhea | very | Giardia |

1-13. (canceled)

14. A composition comprising a glucan encapsulated by a hydrophobic or lipid coating.

15. The composition of claim 14 wherein said glucan comprises a hybrid glucan.

16. The composition of claim 14 wherein said coating comprises essential fat or plant oil.

17. The composition of claim 16 wherein said plant oil comprises soybean oil.

18. The composition of claim 14 further comprising a transfer factor.

19. The composition of claim 18 wherein said transfer factor is encapsulated by a hydrophobic or lipid coating.

20. The composition of claim 19 wherein said coating comprises essential fat or plant oil.

21. The composition of claim 20 wherein said plant oil comprises soybean oil.

22. A composition comprising transfer factor and hybrid glucan.
